(19) United States
(12) Patent Application Publication
(10) Pub. No.: US 2006/0035210 A1
(43) Pub. Date: Feb. 16, 2006

(54) Method of analyzing metabolism for plant, method of producing labeled plants, labeled plants, and method of measuring NMR for plants

(75) Inventors: Kazuo Shinozaki, Tsukuba-shi (JP); Takashi Hirayama, Tsukuba-shi (JP); Jun Kikuchi, Yokohama-shi (JP)

Correspondence Address: FOLEY AND LARDNER LLP;SUITE 500, 3000 K STREET NW, WASHINGTON, DC, 20007, US

(73) Assignee: RIKEN

(21) Appl. No.: 11/083,357

(22) Filed: Mar. 18, 2005

(30) Foreign Application Priority Data
Aug. 13, 2004 (JP) 2004-236188

(51) Int. Cl. C12Q 1/00

(52) U.S. Cl. 435/4

(57) ABSTRACT

The present invention provides a method of analyzing metabolism of a plant in which measurement sensitivity for nuclides such as ¹³C and ¹⁵N having low natural abundance ratios is improved using NMR. A labeled plant uniformly labeled with a stable isotope is produced by providing the plant with a nutrition source labeled with at least one type of stable isotope while growing the plant from a seed or a germinating seedling. The metabolism of a biological substance in the plant is analyzed based on NMR data of a biological substance that contains the stable isotope. The NMR data is acquired by performing NMR measurement on a body of the labeled plant, a portion of the body, or an extract.

### CROSS-REFERENCE TO RELATED APPLICATIONS

The present document incorporates by reference the entire contents of Japanese priority document, 2004-236188 filed in Japan on Aug. 13, 2004.

### BACKGROUND OF THE INVENTION

1. Field of the Invention

The present invention relates to a method of analyzing metabolism for plants using nuclear magnetic resonance (NMR), labeled plants that are used in the method of analyzing, a method of producing labeled plants, and a method of measuring NMR for plants.

2. Description of the Related Art

In the current post-genome age, researches on living organisms based on understanding that the living organism is one form of system are necessary. Metabolomics in which metabolites of a living organism are comprehensively observed is one of such researches. Plants are valuable living organisms that produce organic substances by photosynthesis using solar energy. Plants are positioned at a starting point of the food chain, and all animals, including humans, live by directly or indirectly ingesting the organic substances produced by the plants as nutrient sources. Thus, analyzing the metabolism of plants is remarkably important from viewpoints of food and health care.

Current metabolomics research primarily employs a mass spectrometry (MS), which is a technique for analyzing the metabolism of living organisms using a mass spectrometer. The MS is a method of observing metabolites based on differences in times of flight of compounds. The difference in times attributes to differences in weights of molecules of ionized compounds. Recently, comprehensive analyses have come to be performed using LC-MS, in which liquid chromatography (LC) used for separation purposes is combined with the MS, for the purpose of analyzing metabolic components in a living body roughly classified based on solubility in a solvent, thereby simplifying identification of metabolites and analysis of fluctuations.

However, the MS encounters difficulties in quantitative analysis of mixed samples due to prominent differences in degrees of ionization caused by certain types of compounds referred to as ion suppressors, and the lack of the development of a methodology enabling separation of complex and overlapping signals. In addition, since the MS requires ionization, non-invasive measurement is theoretically impossible, and in vivo measurement is impossible. Furthermore, with the MS, only information on molecular weight and semi-quantitative information on the amounts of substances present, and it is theoretically impossible to use the MS for quantifying molecular mobility.

On the other hand, although analytical methods other than the MS, such as a method of analyzing metabolites using ¹H-NMR, have been reported in recent years (see Non-Patent Literatures 1 to 3), these methods also have inadequate resolution and merely provide statistical analyses of major components based on spectral pattern recognition.
[Non-Patent Literature 1] Defernez, M. and Colquhoun, I. J. (2003) Factors affecting the robustness of metabolite fingerprinting using 1H NMR spectra. Phytochemistry 62: 1009-1017
[Non-Patent Literature 2] Ott, K.-H., Aranibar, N., Singh, B. and Stockton, G. W. (2003) Metabolomics classifies pathways affected by bioactive compounds. Artificial neural network classification of NMR spectra of plant extracts. Phytochemistry 62: 971-985
[Non-Patent Literature 3] Ward, J. L., Harris, C., Lewis, J. and Beale, M. H. (2003) Assessment of 1H NMR spectroscopy and multivariate analysis as a technique for metabolite fingerprinting of Arabidopsis thaliana. Phytochemistry 62: 949-957

However, multidimensional NMR is widely used in the fields of protein identification and structural analysis. Due to its superior reproducibility and quantitative properties, if multidimensional NMR can be applied to metabolomics research, it will be possible to analyze the metabolism of complex mixtures of biological substances that have thus far been difficult to measure. However, the nuclides such as ¹³C and ¹⁵N used in multidimensional NMR have the problem of extremely low detection sensitivity due to low natural abundance ratios thereof.

When measuring NMR of nuclides having low natural abundance ratios, conventionally, such a method has been typically used that a specific compound to be measured is labeled with a stable isotope to improve measurement sensitivity. For example, since the natural abundance ratio of ¹³C is 1.1% and the natural abundance ratio of ¹⁵N is 0.4%, if 100% labeling is possible, it will be possible to observe 100 times the amount of nuclei present in the case of ¹³C, and 250 times the amount in the case of ¹⁵N. An example of a method used to label a specific compound to be measured includes establishing an expression system for a protein to be measured using genetically manipulated escherichia coli, and expressing the protein in a medium containing a nutrient labeled with a stable isotope. Thus, a protein labeled with the stable isotope is obtained.

However, in the field of metabolomics involving the comprehensive observation of the metabolism of living organisms, it is not sufficient to label only a specific compound, but rather all metabolites contained in the organism should be labeled. In this manner, there have been no report of a method for labeling an entire individual plant, and such a method has not yet been established.

### SUMMARY OF THE INVENTION

In view of the above problems, the object of the present invention is to improve detection sensitivity in a method of analyzing metabolism for plants using NMR by uniformly labeling the entire plant.

As a result of assiduous study to solve the above problems, the inventors of the present invention have found that, during the course of growing a plant from a seed or germinating seedling, a labeled plant can be obtained that uniformly incorporates a stable isotope in its body by providing the plant with a nutrient source labeled with at least one type of stable isotope, thereby leading to completion of the present invention on the basis of this finding.

Accordingly, the present invention is summarized as follows:

(1) A method of analyzing metabolism of a plant includes producing a labeled plant that is uniformly labeled with a stable isotope by providing a plant with a nutrition source labeled with at least one type of stable isotope while growing the plant from any one of a seed and a germinating seedling; acquiring information on nuclear magnetic resonance of a biological substance that contains the stable isotope by performing nuclear-magnetic-resonance measurement on any one of a body of the labeled plant, a portion of the body, and an extract; and analyzing metabolism of the biological substance in the plant base on the information.

(2) In the method according to aspect (1), a labeling rate of the labeled plant by the stable isotope is equal to or more than two times of a natural abundance ratio of the stable isotope.

(3) In the method according to aspect (1) or (2), the nutrient source is a carbon source labeled with ¹³C.

(4) In the method according to aspect (3), the plant is an ethylene-insensitive strain.

(5) In the method according to any one of aspects (1) to (4), the nutrient source is a nitrogen source labeled with 15N.

(6) In the method of analyzing metabolism for plants according to any one of aspects (1) to (5), the seed and the germinating seedling are labeled with the stable isotope prior to growing.

(7) A method of producing a labeled plant that is uniformly labeled with a stable isotope includes providing a plant with a nutrition source labeled with at least one type of stable isotope while growing the plant from any one of a seed and a germinating seedling.

(8) A labeled plant that is obtained by the method according to aspect (7).

(9) A portion of a labeled plant that is obtained by the method according to aspect (7).

(10) A method of measuring nuclear magnetic resonance of a plant includes producing a labeled plant uniformly labeled with a stable isotope by providing a plant with a nutrition source labeled with at least one type of stable isotope while growing the plant from any one of a seed and a germinating seedling; and acquiring information on nuclear magnetic resonance of a biological substance that contains the stable isotope by performing nuclear-magnetic-resonance measurement on any one of a body of the labeled plant, a portion of the body, and an extract.

According to the present invention, since a labeled plant can be produced in which a stable isotope is uniformly incorporated in a body thereof, the detection sensitivity can be improved even for the nuclides having low natural abundance ratios, such as ¹³C and ¹⁵N. In addition, the present invention also enables in vivo measurement, which has not been possible with the MS, since the present invention enables non-invasive measurement.

### BRIEF DESCRIPTION OF THE DRAWINGS

FIG. 1 is a schematic for illustrating variation of a ¹³C-HCCH-COSY spectrum of a labeled plant with time;

FIG. 2 is a schematic for illustrating the HCCH-COSY spectrum in each tissue of the labeled plant;

FIG. 3 is a schematic for illustrating a ¹⁵N-HSQC spectrum in each tissue of a labeled plant;

FIG. 4 is a schematic for illustrating a ¹³C-HSQC spectrum of labeled plants;

FIG. 5 is a schematic for illustrating a ¹³C-HSQC spectrum of a wild strain and a ¹³C-HSQC spectrum of an EtOH highly-sensitive strain; and

FIG. 6 is a schematic for illustrating ¹⁵N-HSQC spectra of seeds of labeled plants.

### DETAILED DESCRIPTION OF THE PREFERRED EMBODIMENTS

Exemplary embodiments of the present invention are explained below. In a method of analyzing metabolism of a plant according to the present invention, metabolism of a biological substance in a plant is analyzed by performing NMR measurement on a labeled plant that is uniformly labeled with a stable isotope.

First, the labeled plant used in the NMR measurement is explained. The labeled plant used in the present invention is a plant that is uniformly labeled with a stable isotope. “Uniformly labeled with a stable isotope” refers to incorporation of a stable isotope so that a labeling rate increase for all compounds that contain the stable isotope element among compounds that compose a body of the plant. Since plants grow by superimposing new cells on dead cells, the labeling rates normally differ between an upper portion and a lower portion of the body of the plant. In addition, the labeling rates differ between primary metabolites and secondary metabolites depending on time of metabolizing. However, in the present invention, as long as the labeling rates are increasing for all compounds that compose the plant body that contain the stable isotope element, all the compounds are in a state equivalent to the state of being “uniformly labeled with a stable isotope”.

Such labeled plant can be produced by providing a plant with a nutrient source that has been labeled with at least one type of a stable isotope during the course of growing the plant from a seed or a germinating seedling.

In the present invention, it is possible to uniformly incorporate a stable isotope in an individual body of the plant by providing the plant with the labeled nutrient source at a stage of the seed or the germinating seedling.

There are no particular limitations on a type of the plant to be labeled, and common plants can be used. For example, Arabidopsis thaliana, rice, alfalfa, green bean, soybean, mustard, broccoli, buckwheat, and the like may be used.

During the course of growing the plant, growth of the plant is promoted by providing required nutrient sources to seeds or germinating seedlings. A “nutrient source” refers to all substances essential for plant development. Nutrient sources required for plant development can be broadly classified into CO₂ and H₂O used in photosynthesis, and nutrients such as nitrogen sources, phosphate sources, potassium sources, magnesium sources, calcium sources, trace element sources, and nutriment such as metals.

In the present invention, at least one type of nutrient source selected from among nutrient sources required for the plant development is labeled with a stable isotope and supplied to the plant during the course of growing the plant. The stable isotope used in labeling is a stable isotope of a nuclide for which measurement sensitivity is desired to be enhanced, or a stable isotope for which coupling information can be extracted from a nucleus. For example, the stable isotope includes ²H, ⁷Li, ¹¹B, ¹³C, ¹⁵N, ¹⁷O, ¹⁸O, ²⁹Si, ³³S, ⁴³Ca, ⁴⁷Ti, ⁵⁷Fe, ⁶³Cu, ⁶⁷Zn, ⁷⁷Se, ⁷⁹Br, ¹⁰⁹Ag, ¹¹⁵Sn, ¹²⁹Xe, and ¹⁹⁹Hg. The plant can also be labeled with two or more types of stable isotopes such as ¹³C and ¹⁵N.

Among nutrient sources, CO₂ is a nutrient source that is absorbed through leaves, while nutrient sources other than CO₂ are primarily absorbed through roots. When labeling with a nutrient source that is absorbed through leaves, such as CO₂, the plant should be raised in an atmosphere containing labeled CO₂. When labeling with a nutrient source that is absorbed through the roots, the plant should be raised in a plant bed containing a labeled nutrient source.

When producing a labeled plant with labeled CO₂, it is preferable that a plant is raised in a sealed container to which the labeled CO₂ is supplied. In this case, since ethylene produced by the plant accumulates in the sealed container and the ethylene accumulated sometimes has a detrimental effect on plant development, it is preferable that an ethylene-insensitive mutant strain is used.

In the present invention, it is preferable that the labeling rate of the labeled plant is increased to enhance measurement sensitivity. A preferable labeling rate is dependent on the natural abundance ratio of the stable isotope, and is preferably equal to or more than twice the natural abundance ratio. For example, the labeling rate of ¹³C, which has a natural abundance ratio of 1.1%, is preferably equal to or more than 2.2%, while the labeling rate of ¹⁵N, which has a natural abundance ratio of 0.4%, is preferably equal to or more than 0.8%. More preferably, the labeling rate of the labeled plant is preferably equal to or more than 10 times the natural abundance ratio of the nucleus.

To produce a labeled plant having a high labeling rate, the stable isotope labeling rates for all nutrient sources supplied during the course of plant development should be increased. For example, with respect to those nutrient sources supplied to a plant that contain carbon atoms, although these typically include not only CO₂ absorbed through the leaves, but also carbon atoms present in nutrients absorbed through roots (for example, initial metabolic substances such as glucose and acetates), when labeling the plant with ¹³C, labeling all types of these nutrient sources containing carbon atoms makes it possible to enhance the stable isotope labeling rate for all nutrient sources.

In the present invention, it is preferable that the stable isotope labeling rates is maintained at high rates for all nutrient sources supplied to the plant throughout the entire course of plant development from a seed to a germinating seedling. More specifically, the labeling rates of all nutrient sources supplied to the plant are maintained at equal to or more than twice the natural abundance ratio of the nucleus used for labeling, and preferably maintained at equal to or more than 10 times the natural abundance ratio of the nucleus. There are no particular limitations on the number of times the labeled nutrient source is supplied. The labeled nutrient source may be supplied only once at the beginning, and the label nutrient source may be additionally supplied during the course of development to maintain the labeling rates for all the nutrient sources equal to or greater than a predetermined value.

Moreover, another preferable method of producing a labeled plant includes a method using a seed (hereinafter, “labeled seed”) labeled in advance with a stable isotope or a germinating seedling that has germinated from the labeled seed. Use of such labeled seed or germinating seedling germinated from the labeled seed makes it possible to further increase the labeling rate of the labeled plant. The labeled seed can be obtained from, for example, a grown labeled plant.

Information on nuclear magnetic resonance of biological substances containing a stable isotope is then acquired by performing NMR measurement on the labeled plant obtained. In the present invention, biological substances to be subjects of analysis of metabolism are the biological substances that are labeled with the stable isotope.

First, a sample for the NMR measurement is prepared from the labeled plant obtained. An individual labeled plant itself can be used as the sample for the NMR measurement, and a portion (leaf, flower, stem, root, seed, etc.) of the labeled plant or an extract obtained from the labeled plant can also be used for the sample for the NMR measurement.

The NMR measurement is then performed on the sample for NMR measurement obtained from the labeled plant to acquire the information on nuclear magnetic resonance of the biological substances containing the stable isotope. There are no particular limitations on methods of the NMR measurement, and common measurement methods can be used, and both one-dimensional observation methods and multi-dimensional observation methods can be used. For example, by combining various multi-dimensional observation methods for assigning chemical shifts (for example, DQF-COSY, TOCSY, NOESY, INADEQUATE, NOESY-HSQC, TOCSY-HSQC, HCCH-TOCSY, or HCCH-COSY), signals can be assigned more systematically. In other words, a measurement method suitable for a metabolite desired to be observed should be selected from among the common NMR measurement methods.

According to the present invention, various pieces of metabolic information on biological substances can be obtained by using the NMR. The information obtained by the NMR includes information on a type of compound (chemical shift) and its amount (peak intensity). In addition, information on mobility of a substance in the body can be obtained from a line width and intensity of a spectrum. Furthermore, nuclear magnetic imaging makes it possible to obtain an image that illustrates distribution of the biological substances within the body of the labeled plant.

To analyze the metabolism of the biological substances in a plant, information on the nuclear magnetic resonance should be acquired for two or more samples having different metabolic conditions, and difference in the information between the samples should be analyzed. “Different metabolic conditions” includes not only a case of having different metabolic conditions due to a time course of metabolism in the same individual caused by changes in an environment surrounding the plant as well as growth of the plant itself, but also a case of different metabolic conditions at different sites in the same individual as well as a case of different metabolic conditions between genetically different individuals. For example, how stress affects a plant internally can be determined by applying stress to the labeled plant and then analyzing quantitative changes in metabolites before and after applying the stress based on the differential spectrum. In addition, what kind of the biological substance increases or decreases over time during the course of plant growth can be analyzed by observing quantitative changes in metabolites during the course of the plant growth including stages such as germination, leaf deployment, and flowering. Furthermore, what kind of genetic difference affects expressed proteins can be determined by respectively producing labeled plants for wild strains and mutant strains of a plant, and then analyzing differences in types and amounts of proteins expressed in the wild strain and the mutant strain.

As explained above, according to the present invention, since a labeled plant can be produced in which a stable isotope is uniformly incorporated in a body of a plant, detection sensitivity can be improved even for nuclides such as ¹³C and ¹⁵N that have low natural abundance ratios, and coupling information between nucleus spins can be obtained. Furthermore, widening of 1H signals due to anisotropy of 1H-1H dipole interaction intrinsic to biological samples can be prevented by uniformly labeling plants with a stable isotope. In addition, since the present invention allows non-invasive measurement, measurements can be performed in vivo, which has not been possible with the MS.

### EXAMPLES

The present invention is explained in more detail based on examples below. Note that the present invention is not limited to the examples below.

### Example 1

### Production of a Labeled Plant

A labeled plant uniformly labeled with a stable isotope was prepared for Arabidopsis thaliana. An ethylene-insensitive mutant strain ein2-5 was used for a label for Arabidopsis thaliana.

The mutant strain ein2-5 of Arabidopsis thaliana, variety Columbia was seeded in a plant bed that includes vermiculite and perlite (50% each (volume/volume)), and was maintained at 4° C. for 3 days to 4 days to promote germination. Germinating seedlings of the mutant strain ein2-5 were raised at 22° C. to 23° C. using a light-dark cycle of 16 hours of daylight and 8 hours of night. During the course of development, nutrient salts of the composition indicated below (all concentrations are final concentrations) were provided once a week.

| | | |
|---|---|---|
| KNO₃ | 5 | millimoles (mM) |
| KPO₃ | (pH 5.5) 2.5 | mM |
| MgSO₄ | 2 | mM |
| CaCl₂ | 2 | mM |
| Fe EDTA | 50 | micromoles (μM) |
| H₃BO₃ | 70 | μM |
| MnCl₂ | 14 | μM |
| CuSO₄ | 0.5 | μM |
| ZnSO₄ | 1 | μM |
| NaMoO₄ | 0.2 | μM |
| NaCl | 10 | μM |
| CoCl₂ | 10 | nanomoles (nM) |

(1) Production of a ¹³C-Labeled Plant

Labeling with ¹³C was carried out by preparing a sealed acrylic chamber, filling the chamber with ¹³CO₂ at 340 parts per million (ppm), and growing a plant inside the chamber. The atmosphere in the chamber was replaced every 2 to 3 days by pumping in 340 ppm of ¹³CO₂ for ventilation. A ¹³C labeling rate of the labeled plant after 30 days of being raised was about 30%.

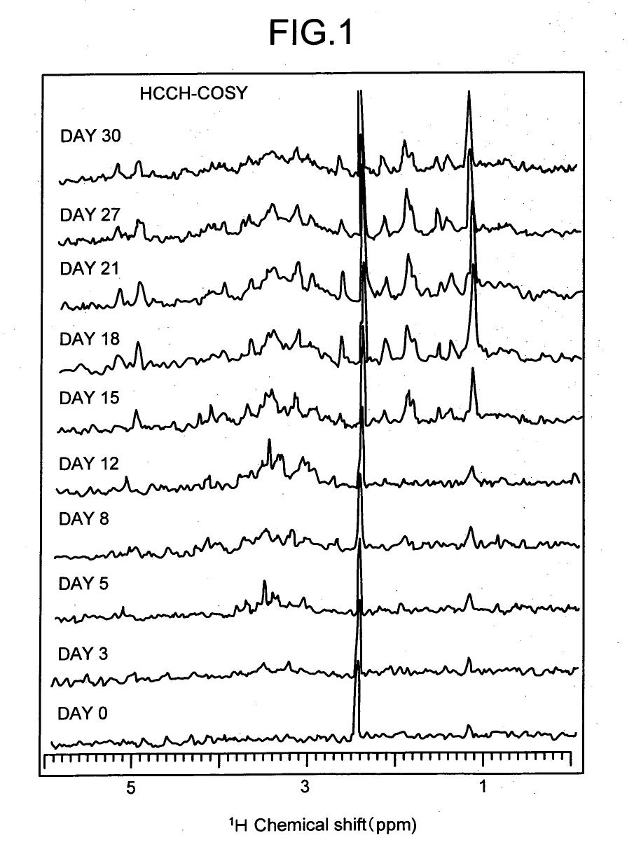

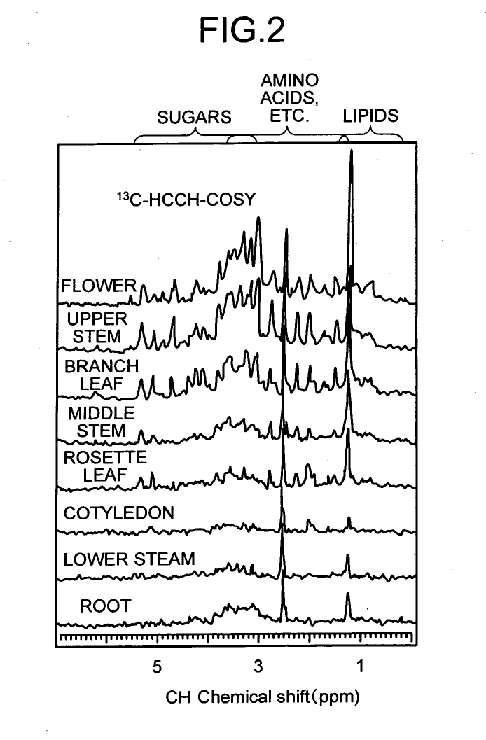

Variation of an HCCH-COSY spectrum of a labeled plant with time is shown in FIG. 1. It is possible to observe that ¹³C was gradually incorporated into a body of the plant as days increases for growing the plant. In addition, a ¹³C-HCCH-COSY spectrum in each tissue of the labeled plant is shown in FIG. 2. It is possible to observe that ¹³C was incorporated in various compounds of each tissue.

(2) Production of a ¹⁵N-Labeled Plant

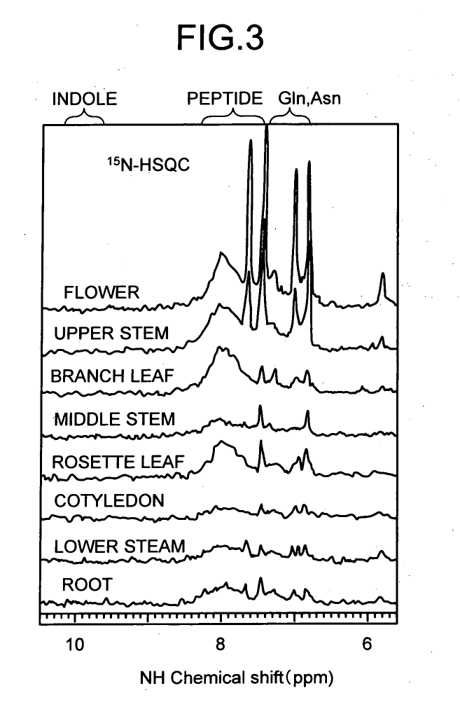

In a case of ¹⁵N labeling, a ¹⁵N-labeled plant was produced by providing ¹⁵KNO₃ instead of a nutrient salt KNO₃ described above until the plant produced seeds and died. A ¹⁵N labeling rate of the labeled plant after 30 days of growing was about 98%. A ¹⁵N-HSQC spectrum in each tissue of the labeled plant is shown in FIG. 3. It is possible to observe that ¹⁵N was incorporated in various compounds of each tissue.

In addition, it was confirmed that uniformly labeled plants were also able to be produced using rice, alfalfa, green bean, soybean, mustard, broccoli, and buckwheat by the technique applied to Arabidopsis thaliana.

### Example 2

### Production of a Labeled Plant in Agar Medium

Sterilized seeds were disseminated in MS medium (containing mixed salts for Murashige and Skoog medium, 1% (weight (w)/volume (v)) ¹³C uniform labeled glucose, 0.5 mM MES (pH 5.8) and 0.8% (w/v) agar) and raised for 2 weeks in a dark location following germination. It was confirmed that ¹³C was able to be uniformly incorporated in each tissue by ¹³C-HCCH-COSY. A ¹³C labeling rate of the labeled plant after 30 days of being raised was about 95%.

### Example 3

### ¹³C-HSQC Measurement Using the ¹³C-Labeled Plant

(1) In Vitro NMR Sample Preparation

First, the ¹³C-labeled plant (Arabidopsis thaliana) obtained in Example 1 was rapidly frozen with liquid nitrogen to interrupt biological activity of the ¹³C-labeled plant. Subsequently, a moisture-containing sample spontaneously melted was weighed out to 2 milligrams (mg) to 100 mg. 0.5 milliliter of NMR solvent indicated below was added to the sample. The sample was completely crushed with a mortar and transferred to an eppendorf tube. Deuterium-loaded dimethyl sulfoxide capable of dissolving both hydrophilic and hydrophobic molecules was used for the solvent. The solvent of the sample in the eppendorf tube was centrifuged for 5 minutes at 15,000 grams, and a precipitate was removed. The sample was placed in an NMR tube having a diameter of 5 millimeters as an NMR sample.

(2) Measurement by Multidimensional NMR

Multidimensional NMR measurement was performed using a Bruker 500 MHz-NMR spectrometer and a trinuclear probe equipped with a triaxial gradient. Two-dimensional ¹³C-HSQC measurement was performed by removing water signals contained in a large amount in the sample using a water-flip-back method (Grzesiek & Bax, 1993) and typically measuring by integrating 16 times to 160 times for 1024 points (f2 axis)×128-256 points (f1 axis). Free induction decay (FID) data was processed by performing a fourier conversion using an nmrPipe program (Delagrio, et al, 1995), and a zero filling processing. To acquire a two-dimensional differential spectrum, macro of the nmrPipe program was edited.

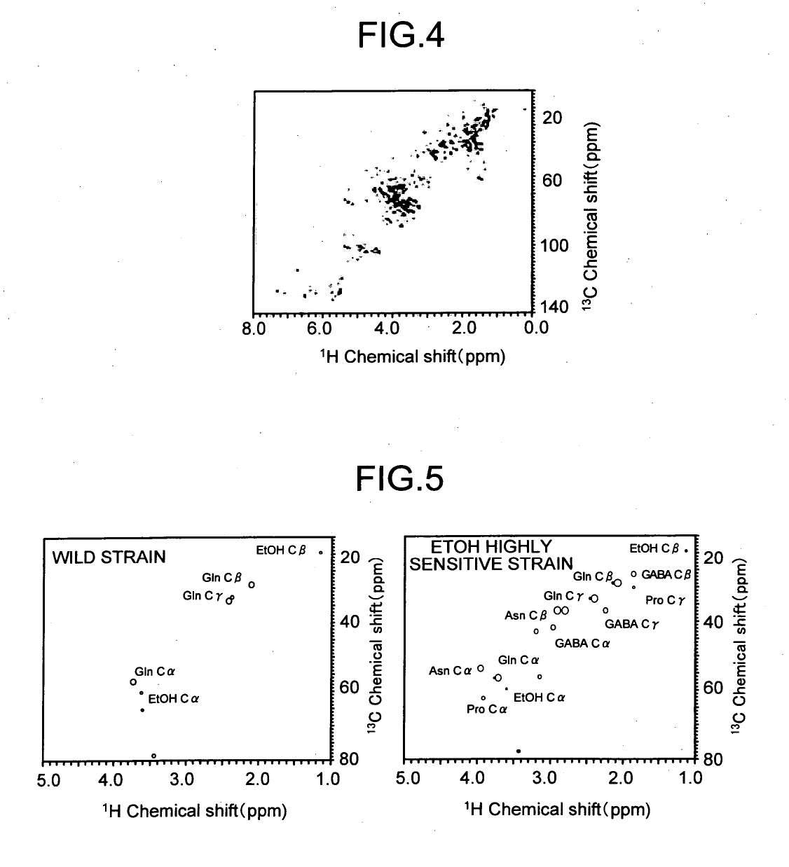

A two-dimensional ¹³C-HSQC spectrum obtained is shown in FIG. 4. In an example shown in FIG. 4, 477 cross signals were observed in a case of extracting peaks with an nmrPipe software. Since coupling with hydrogen covalently bonded to carbon is observed in the ¹³C-HSQC method, although three signals corresponding to carbons Cα, Cβ, and Cγ are observed, for example, in a case of Gln, only two are observed in a case of Asn. Major metabolites present at high concentrations frequently have simple chemical structures, and if two to three signals are presumed to be obtained on an average for a single compound, it is believed that roughly 150 to 200 metabolites can be observed in the ¹³C-HSQC spectrum.

### Example 4

### EtOH Stress Response of the ¹³C-Labeled Plant

The ¹³C-labeled plant was produced in the same manner as Example 1 for a wild strain and an EtOH highly-sensitive strain of Arabidopsis thaliana. An in vitro NMR sample was prepared in the same manner as Example 2 to obtain the ¹³C-HSQC spectrum.

FIG. 5 depicts difference in a differential spectrum between the wild strain (left) and the EtOH highly-sensitive strain (right) for a purpose of analyzing changes in metabolites during response to EtOH stimulation. In this case, the (¹³C-HSQC spectrum when EtOH is added) was subtracted from the (¹³C-HSQC spectrum when normally cultivated). In FIG. 5, those signals that decreased due to addition of EtOH are indicated with positive contour lines (black dots), while those signals that increased are indicated with positive contour lines (circles). In the EtOH highly-sensitive strain, in addition to Gln, amino acids such as Asn, Pro, and gamma-aminobutyric acid (GABA) were newly synthesized from ¹²C EtOH as compatible solutes during response to stress, and positive contour lines appeared due to a decrease in the ¹³C signals. This unique result indicates that with the present method, it is possible to not only obtain information merely indicating changes in an amount of metabolites, but also determine new metabolites secondarily synthesized in response to stimulation by compounds such as EtOH. Uniform labeling is beneficial also in this point.

### Example 5

### Light Environment Response of a ¹⁵N-Labeled Seed

(1) In Vivo NMR Sample Preparation

To measure a ¹⁵N-labeled seed in vivo, 180 microliters of distilled water containing 10% heavy water were added directly to a dried seed, and then immediately transferred to an aqueous solution reaction microtesttube (Shigemi) using a pasteur pipette to be used as an NMR sample.

(2) Multidimensional NMR Measurement for In Vivo Samples

In order to measure a ¹⁵N-labeled seed in vivo, an optical fiber having a length of 2 meters (m) was inserted from a halogen light source (Moritex, MHF-D100LR) into an NMR superconducting magnet, and kinetic changes in ¹⁵N metabolism during germination in vivo was measured while changing light intensity from 0 μmol/m2/s to 50 μmol/m²/s and temperature from 277K to 295K to observe response to a light environment. The ¹⁵N-HSQC spectrum was acquired using measuring instrument and data processing similar to those in Example 2.

(3) Examples of In Vivo Measurement of Plant Seeds

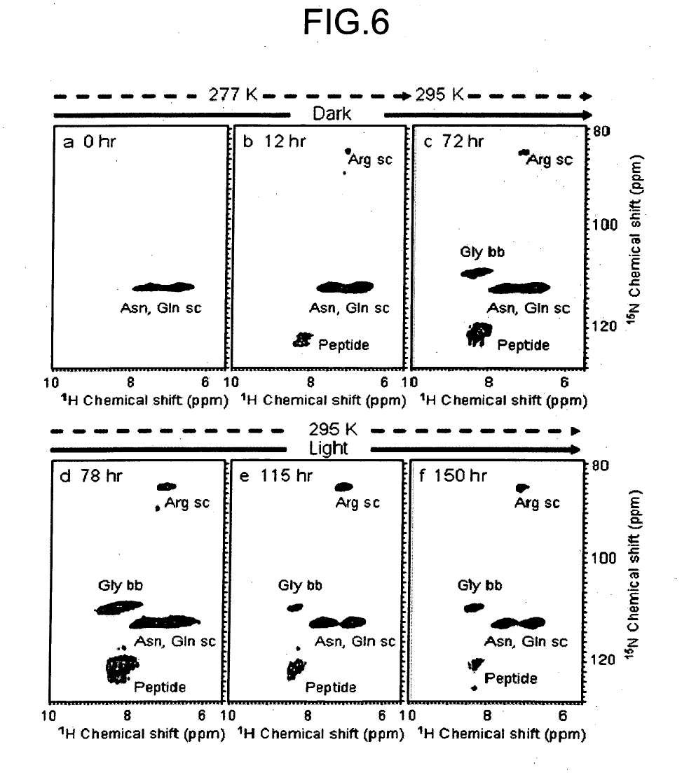

FIG. 6 depicts the in vivo 15N-HSQC spectra of 15N-labeled seeds. Only coupling between nitrogen and hydrogen is observed in the form of cross signals in the 15N-HSQC spectra. Four types of four-dimensional information can be obtained from these spectra that can be broadly classified as A: mobility of compounds in a living body (line width and intensity), B: types of compounds (chemical shift values), C: amounts (intensity), and D: metabolic kinetics from time-based observations (time course of chemical shifts and intensities). As shown in FIG. 6, first, the seed is in a process of absorbing moisture at point a (0 hour). Since this is not satisfactory even for low molecular weight mobility, only side chain signals of Asn and Gln can be observed. The side chain signal of Arg begins to be observed at point b (12 hours). Furthermore, the signal of a main peptide chain begins to become distinct corresponding to rise in system temperature at point c (72 hours), and corresponding to irradiation of light by the optical fiber device inserted into the NMR magnet, the amount of peptide can be determined to reach a maximum at point d (78 hours) based on increase in signal intensity. Furthermore, when germination begins and protein synthesis becomes active as a result of irradiation of light, peptides and amino acids are used as materials for protein, and signal intensity continues to decrease due to the formation of high molecular weight proteins having low mobility (point e (115 hours), point f (150 hours)).

As described above, the present invention is useful for analyzing metabolism of a plant using NMR, and can be particularly applied to discover metabolic abnormalities in a plant and to determine a cause of the abnormalities, or to an applied research such as growth monitoring and quality control in fields of metabolic engineering and agriculture, analysis of effects of agricultural chemicals, food quality control, nutritional control, and quality control of gene recombinant crops, as well as basic research such as simulation of life-sustaining activities in fields such as system biology.

1. A method of analyzing metabolism for a plant, the method comprising:
producing a labeled plant that is uniformly labeled with a stable isotope by providing a plant with a nutrition source labeled with at least one type of stable isotope while growing the plant from any one of a seed and a germinating seedling;
acquiring information on nuclear magnetic resonance of a biological substance that contains the stable isotope by performing nuclear-magnetic-resonance measurement on any one of a body of the labeled plant, a portion of the body, or an extract; and
analyzing metabolism of the biological substance in the plant base on the information.

2. The method according to claim 1, wherein a labeling rate of the labeled plant by the stable isotope is equal to or more than two times of a natural abundance ratio of the stable isotope.

3. The method according to claim 1, wherein the nutrient source is a carbon source labeled with ¹³C.

4. The method according to claim 3, wherein the plant is an ethylene-insensitive strain.

5. The method according to claim 1, wherein the nutrient source is a nitrogen source labeled with ¹⁵N.

6. The method according to claim 1, wherein the seed and the germinating seedling are labeled with the stable isotope prior to growing.

7. A method of producing a labeled plant that is uniformly labeled with a stable isotope, the method comprising providing a plant with a nutrition source labeled with at least one type of stable isotope while growing the plant from any one of a seed and a germinating seedling.

8. A labeled plant that is uniformly labeled with a stable isotope, wherein the labeled plant is obtained by providing a plant with a nutrition source labeled with at least one type of stable isotope while growing the plant.

9. A portion of a labeled plant that is uniformly labeled with a stable isotope, wherein the labeled plant is obtained by providing a plant with a nutrition source labeled with at least one type of stable isotope while growing the plant.

10. A method of measuring nuclear magnetic resonance of a plant, the method comprising:
producing a labeled plant that is uniformly labeled with a stable isotope by providing a plant with a nutrition source labeled with at least one type of stable isotope while growing the plant from any one of a seed and a germinating seedling; and
acquiring information on nuclear magnetic resonance of a biological substance that contains the stable isotope by performing nuclear-magnetic-resonance measurement on any one of a body of the labeled plant, a portion of the body, and an extract.
